(19) United States
(12) Patent Application Publication
(10) Pub. No.: US 2004/0073139 A1
(43) Pub. Date: Apr. 15, 2004

(54) Cannula for extracting and implanting material

(76) Inventors: Joshua A. Hirsch, Boston, MA (US); Scott H. McIntyre, San Jose, CA (US); Yves P. Arramon, Capitola, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/269,926

(22) Filed: Oct. 11, 2002

(51) Int. Cl. A61B 10/00

(52) U.S. Cl. 600/564

(57) ABSTRACT

Devices and methods for performing medical procedures at a target site within the body of a patient. Such procedures include the extraction of bone or tissue samples from a target site and the delivery a filler material to the target site. The devices include an outer cannula for accessing the target tissue or bone area, a stylet for directing and delivering the outer cannula, and an inner cannula for tissue retrieval or removal. Both the stylet and inner cannula are individually sized and configured for slidable insertion and delivery into the outer cannula. The outer cannula may also be employed for the delivery of a filler material to within the bone or tissue target site.

### FIELD OF THE INVENTION

The present invention generally relates to cannulae for use in extracting bone or soft tissue from a target site and/or for injecting or implanting other material into that site.

### BACKGROUND OF THE INVENTION

The removal or extraction of bone or tissue from a human or an animal is often done for pathological evaluation of the bone or soft tissue to verify a diagnosis, assess the extent of a condition, and, particularly for subjects undergoing radiation and chemotherapy treatment, to evaluate the extent of damage of these therapies. Bone marrow is also extracted or harvested from a donor for transplantation into a patient. Additionally, bone marrow extraction is sometimes performed to create a space within the bone's medullary space into which an implant material, i.e., bone “cement,” is injected. Such a procedure is used in the context of a vertebroplasty in which the cancellous bone of the vertebrae is supplemented with polymethylmethacrylate (PMMA) or another filler material in order to stabilize the spine.

The extraction procedure is typically performed by means of a biopsy needle or cannula which is manually or automatically inserted into the target area of bone or tissue, and then removed with a “core” of bone or tissue obtained within the lumen of the needle or cannula. This procedure can be very painful to the patient. Particularly when taking a biopsy of bone, the biopsy needle may need to be twisted or rotated to separate the sample from the surrounding bone marrow, further increasing the pain experienced by the patient.

Moreover, due to the configuration of prior art devices, it can be difficult to obtain a sufficient amount of sample. Even if a sufficient amount of sample is obtained within the biopsy needle, the needle configuration is such that it is difficult to retain the cored sample within the biopsy needle during removal of the biopsy needle from the target site. As such, it is often necessary to repeat the extraction or coring procedure. Due to the repetitive and aggressive movement of the biopsy needle, the cored sample, if actually retained within the biopsy needle, is often crushed making analysis of the sample more difficult.

U.S. Pat. No. 6,416,484 to Miller et al. describes a bone marrow biopsy assembly which includes an outer cannula or biopsy needle having a tapered distal end, and an extractor having an inner cannula sized for slidable insertion into the proximal end of the outer cannula. The inner cannula has a distal working end having a diameter larger than the diameter of the tapered distal end of the outer cannula. The distal working end is provided with a cutting head having a cutting tip which is hinged to the inner cannula. The hinge is deformable to allow the cutting head to bend when pushed into the tapered distal end of the outer cannula thereby forcing the cutting tip to sever the tissue sample from the tissue bed. In use, the outer cannula remains in place while multiple extractions may be performed using the inner cannula. Maintaining the position of the outer cannula avoids the need to make another hole in the bone cortex.

U.S. Pat. No. 6,086,543 to Anderson et al. describes a core biopsy cannula for retrieving specimens of soft tissue. The cannula has a sharpened conical tip and a tissue urging feature in the form of spiral thread or a plurality of slots recessed within the inner surface of the cannula lumen. Such a feature helps to draw the tissue specimen cut by the conical tip into the lumen as the cannula is rotated and helps in retaining the collected specimen within the lumen as the cannula is retracted. When the cannula is rotated, the spiral thread or other recess or slots in the inner cannula surface causes the tissue entering the lumen to become twisted and somewhat compressed and to advance in a proximal direction as it is twisted into a generally helical shape within the lumen. When rotation of the cannula is ceased, the compressed tissue tends to untwist and decompress or expand, thereby exerting a radially directed force against the interior wall of the lumen, which, as the patent asserts, is more likely to retain the specimen in the cannula lumen as the cannula is removed from the tissue bed.

The present invention teaches another assembly and approach to extracting samples from bone and tissue. The approach advantageously offers an elegant assembly which does not require the use of moving or hinged components. Further, the inventive assembly is configured so as to more easily obtain a sufficient sample core and to ensure retention of the sample core upon removal from the patient. Additionally, the present invention is very versatile in that it is usable with bone as well as soft tissue applications, and optionally serves as a conduit for implanting material into a site from which bone or tissue has been removed.

### SUMMARY OF THE INVENTION

The present invention provides devices and methods for the extraction of bone or tissue samples from the body of a patient. Such devices are distinguished from certain known biopsy systems that provide more complex mechanisms to obtain a suitable biopsy sample. The invention is further distinguishable from prior art systems in that the inventive system may also serve as a delivery system for the implantation of filler materials within the bone or tissue.

The present invention preferably includes an outer or “docking” cannula for accessing the target tissue or bone area, a stylet for directing and delivering the outer cannula, and an inner or “extraction” cannula for biopsy retrieval or bone/tissue removal. Both the stylet and inner cannula are individually sized and configured for slidable insertion and delivery into the outer cannula. The inner cannula has a particularly configured distal portion having an inwardly beveled or tapered leading edge defining a frustro-conical or truncated funnel or cone shape. The outer cannula is also employed for the delivery of a filler material to the bone or tissue subsequent to bone or tissue removal. In particular, the combination of the inventive elements is well-suited for vertebroplasty applications.

In use, the outer cannula (usually with the stylet) provides a desired straight-line access path through hard bone or soft tissue. Once such access is established, the stylet is removed from the outer cannula and the inner cannula is delivered through the outer cannula to the target access site. The inner cannula is then used to obtain a core of the bone or tissue accessed, which cored material may be used as a sample for analysis or may be discarded if being replaced by a synthetic implant material. The coring process may be repeated as necessary using the same inner cannula or multiple inner cannulas while maintaining the original placement of the outer cannula. In applications involving the implantation or delivery of material to within the accessed site, the inner cannula is removed and flowable material or a medical device for delivering a flowable material, or the like, may be introduced through the outer cannula. One particular methodology of the present invention is use of the assembly or system for performing a vertebroplasty procedure and use of such auxiliary equipment as described below or otherwise available.

Other features, aspects and variations of the invention will become apparent to those skilled in the art upon reading this disclosure in combination with the accompanying figures.

### BRIEF DESCRIPTION OF THE FIGURES

To facilitate understanding, the same reference numerals have been used (where practical) to designate similar elements that are common to the Figures. Some such numbering has, however, been omitted for the sake of drawing clarity.

FIGS. 1A and 1B illustrate an assembly or system of the present invention wherein FIG. 1A illustrates an outer cannula and a stylet of the present invention operatively engaged and FIG. 1B illustrates an inner cannula of the present invention.

FIG. 2A is a cross-sectional view of the distal end portion of the outer cannula and stylet of FIG. 1A.

FIG. 2B is a cross-sectional view of the distal end portion of the inner cannula of FIG. 1B operatively engaged within the outer cannula.

FIG. 3 illustrates a cement injection system in operative use with the outer cannula of the present invention.

FIG. 4 is a photograph illustrating a comparison of samples of cored cancellous tissue obtained by the inner cannula of the present invention and prior art cannulas.

### DETAILED DESCRIPTION OF PREFERRED EMBODIMENTS

In further describing the subject invention, the subject devices and systems will be described first followed by a description of the subject methods and a summary of the kits which include the subject devices for performing the subject methods.

Before the present invention is described in detail, it is to be understood that this invention is not limited to particular variations set forth herein as various changes or modifications may be made to the invention described and equivalents may be substituted without departing from the true spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs.

Definitions

The terms “bone” and “tissue” are used herein interchangeably unless more specifically defined, for example, as “bone marrow,” “soft tissue,” etc.

The term “implant material” or “filler material” as used herein means any material implanted into or used to fill in a space within the bone or tissue or used to augment the currently existing bone or tissue. Polymethylmethacrylate (PMMA) is an example of such a filler material used to fill in or supplement cancellous bone tissue.

The terms “cannula” and “needle,” with respect to defining or describing the present invention, may be used interchangeably herein.

The term “inviolate” when referring to the present invention, such as to a surface of a cannula of the present invention, may be used interchangeably to mean a continuous, smooth surface without protrusions or grooves.

The terms “conical,” “frustum” and “frustro-conical” all generally refer to the same general shape and may be used interchangeably herein.

Devices and Systems

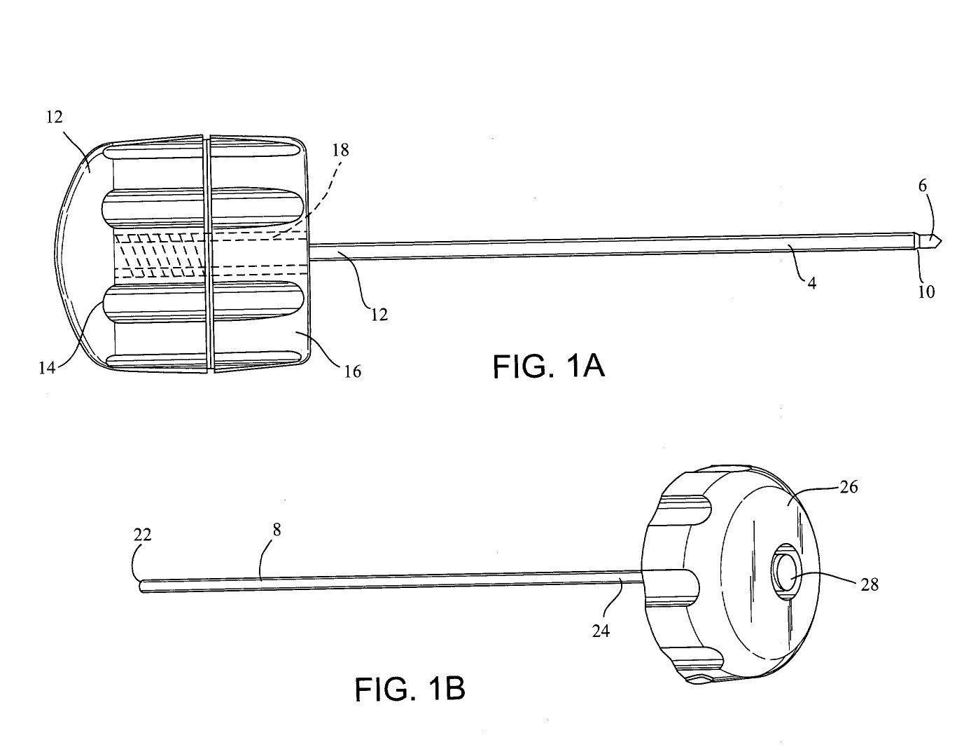

Referring now to the Figures, there is shown a system of the present invention which generally includes an outer or “docking” cannula 4, a stylet 6 and an inner or extraction cannula 8. In FIG. 1A, outer cannula 4 and stylet 6 are operatively assembled. Inner cannula 8 is also operatively engageable with outer cannula 4 in the same manner as stylet 6. Both the stylet and inner cannula are sized and configured for interchangeable insertion into a proximal end of outer cannula 4. Preferably, all three components are made of a substantially rigid material such as a ceramic or a metal such as surgical grade stainless steel, titanium, titanium alloys or the like.

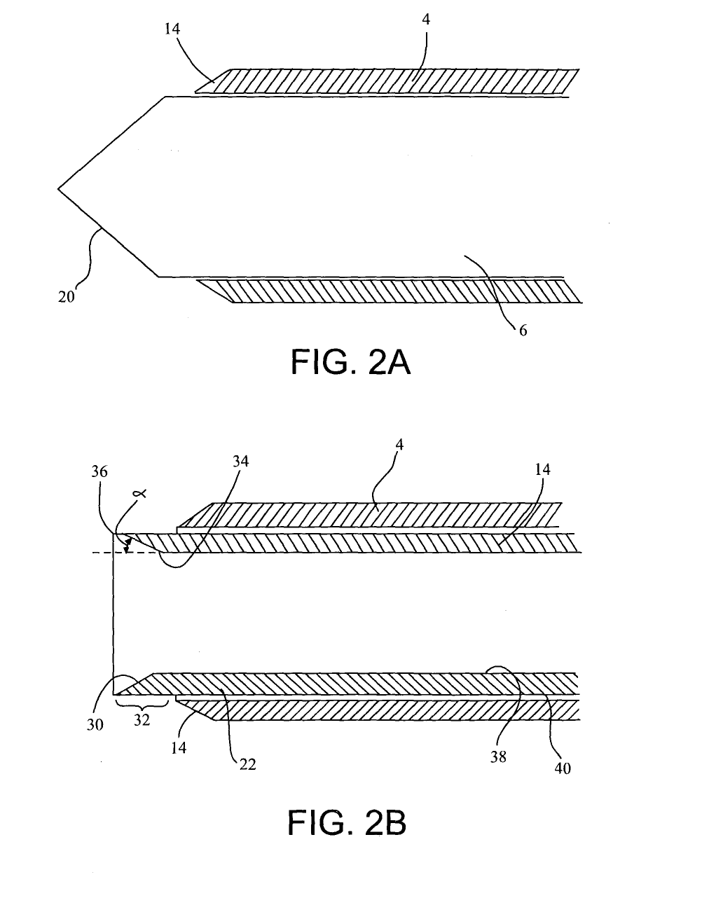

Outer cannula 4 defines a hollow lumen extending from a distal end 10 to a proximal end 12. Distal end 10 may terminate in a tapered or outwardly beveled distal tip 14 (see FIGS. 2A and 2B) for facilitating penetration into the bone or tissue of the target access site. A handle 16 is provided at the proximal end 12 of outer cannula 4 which facilitates the user's handling and manipulation of the cannula. Extending proximally from handle 16 is a threaded extension or connector 18 (shown in phantom in FIG. 1A) for locking onto the respective handles of stylet 6 and inner cannula 8. Extension or connector 18 is also adapted for engagement with a system for the controlled injection of flowable material, such as PMMA. Such systems which may be used with the present invention are disclosed in U.S. patent application Ser. No. 09/408,690 filed on Sep. 30, 1999, entitled “High Pressure Delivery System,” incorporated herein by reference.

As shown in FIG. 2A, stylet 6 includes a solid shaft which may provide a sharp distal tip 20 for facilitating the penetration of outer cannula 4 into the target site. Distal tip 20 may have any configuration suitable for the application at hand. Exemplary configurations include threaded, conical, pyramidal, triangular, beveled and double beveled. Affixed to the proximal end of stylet 6 is a domed handle 12 having a threaded receptacle 14 for receiving and locking to extension 18 of outer cannula handle 16. Collectively, when operatively coupled, outer cannula handle 16 and stylet handle 12 provide an ergonomically designed handle which may be easily grasped and rotated by the user. The domed head of the coupled handles also provides a sufficient surface area to which the user may apply a force in order to further penetrate the assembly into a target site.

Inner cannula 8 defines a hollow lumen extending from a distal end 22 to a proximal end 24. Affixed to the proximal end 24 is a domed handle 26 which also has a threaded receptacle (not shown) for receiving and locking to extension 18 of outer cannula handle 16. The coupled handles provide the same ergonomic advantages as described above with respect to the coupling of the inner cannula and stylet handles. Within inner cannula handle 26 and in fluid communication with the lumen of cannula 8 is threaded exit port 28 which allows for a tissue core to exit cannula 8 as it is progressively advanced proximally through the cannula's lumen. The port's threaded configuration allows it to be optionally attached to a source of negative pressure to facilitate extraction of the cored sample from cannula 8 as well as to a syringe for injection contrast material or saline, as described below.

As shown in FIG. 2B, distal end 22 of the inner cannula 8 has a distal portion 32 having an inwardly beveled or tapered configuration defining a leading surface 30. Such a configuration preferably defines a funnel, conical or frustroconical shape. The taper extends from an outer diameter or distal edge 36 to an inner diameter or proximal edge 34, forming an angle α with the longitudinal axis of the cannula. Angle α is generally in the range from about 5° to about 60°, typically in the range from about 10° to about 20°, and is more typically about 15°. Tapered leading surface 30 advantageously provides for a tissue core to be cut having a relatively large diameter and then advantageously compresses the tissue as it advances proximally along leading surface 30. The greater the angle α, the more the cored tissue is compressed as it approaches proximal edge 34. As the cored sample enters the proximal or straight gauge portion of the cannula's lumen, the compressed tissue remains compressed until it is extracted from the proximal end or ejected out of the distal end. Unlike the biopsy cannula of the '543 patent discussed above, the cored tissue is continuous compressed while in the lumen of the cannula. This continuous compression helps in retaining the collected specimen within the lumen as the cannula is retracted from the tissue bed.

Another aspect of inner cannula 8 is that its outer diameter is constant along its length from the proximal end 24 to the distal tip 22 and the inner diameter is constant along its length from the proximal end 24 up to but not including the distal tip 22. In other words, within a distal portion 32 of inner cannula 8, the inner beveled portion defines a proximal edge 34 and a distal edge 36. Inner cannula 8 has a constant outer diameter from proximal end 24 to the distal edge 36, and the cannula's wall thickness is constant from proximal end 24 to the proximal edge 34 of distal portion 32 and tapers inwardly from the distal edge 36 to the proximal edge 34. Thus, at the portions where the wall thickness is constant, the inner luminal surface 38 and the outer surface 40 of cannula 8 are parallel to each other. Another aspect of the inner cannula is that distal edge 36 defines a plane which is at least substantially normal to the longitudinal axis of inner cannula 8. In one embodiment, distal edge 36 is substantially circular thereby defining a plane which is perpendicular to the longitudinal axis of cannula 8.

The dimensions of the various components of the present invention are dictated by the application in which they are used. Typical ranges are provided as follows. Outer cannula 4 preferably has a gauge in the range from about 9 Ga to about 13 Ga, and is about 11 Ga for a vertebral application, for example. The length of outer cannula 4 is in the range from about 6 cm to about 20 cm, and more typically from about 10 cm to about 15 cm. Inner cannula 8 preferably has a gauge in the range from about 11.5 Ga to about 16 Ga, and is typically about 13.5 Ga for a vertebral application, for example. The size of the inner cannula is smaller than the outer cannula in any assembly of the present invention. The length of inner cannula 8 is in the range from about 6 cm to about 21 cm, and more typically is in the range from about 10 cm to about 16 cm. Stylet 6 has a length dimension slightly greater than that of outer cannula 4 such that distal tip 20 extends from distal end 14 of outer cannula 4. The diameter of stylet 6 is sized slightly less than the inner diameter of outer cannula 4.

Methods

The methods of the present invention involve the extraction or removal of bone or tissue at a target site in the body. Certain of these methods further involve the delivery of filler material into the target site. Suitable applications of the subject methods include intravertebral vertebroplasty and mandibular and hip augmentation. For purposes of further describing the present invention, the subject methods are described in the context of an intravertebral vertebroplasty procedure; however, such is not intended to be limiting in any way to the invention as this invention is useable in a myriad of other procedures where tissue is to be removed and/or flowable material is to be delivered to within the bone or tissue of a patient.

Initially, the surgeon or user identifies an external landmark which is to be penetrated in order to access the target area within the patient. Such identification process may be visually assisted by fluoroscopy or other imaging techniques known in the surgical arts. Next, an injection is given to anesthetize the skin and subdermal tissue where the insertion will occur. A long needle, having a length sufficient to access the periosteum of the target vertebrae is then used to inject an anesthetic.

After sufficient time has passed to effectively anesthetize the skin, the surgeon inserts the distal ends of the combined outer cannula 4 and stylet 6 assembly (as illustrated in FIG. 1A) through the skin at the identified landmark. Alternatively, an incision may first be initiated with a scalpel. With a suitable amount of force exerted on the assembled handles 12 and 16, the assembly is advanced using a translation motion until distal tip 20 of stylet 6 abuts the cortical bone of the target vertebra or the periosteum surrounding it. Optionally with the aid of medical imaging, outer cannula/stylet assembly is positioned with respect to the pedicle of the vertebra at the desired orientation for passing therethrough and into the body of the vertebra. The operator may then cause the assembly to penetrate the target site by rotating, pushing and/or torquing the assembled handle such that stylet 6 enters into the vertebral body. Such actions are continued until the assembly is advanced to a desirable location and depth. Upon achieving the desired placement of outer cannula 4, the operator reverse rotates the stylet 6 and, thus, unthreads stylet handle 12 from outer cannula handle 16 while preventing rotation of cannula 4.

Once stylet 6 has been completely unthreaded and removed from outer cannula 4, fluoroscopic imaging/viewing of the placement of outer cannula 4 may optionally be performed to assure that the cannula did not move during removal of stylet 6. Optionally, a contrast agent may be injected through the cannula and the flow of the contrast agent is viewed fluoroscopically or with other imaging in order to ascertain that the tip of the cannula has not been placed in a vein or other significant vessel. After completing the flow of the contrast agent, the remnants of the contrast agent are flushed out of the target site by injecting a flushing solution (e.g., saline) through the cannula 4 using a syringe or other injector.

Inner cannula 8 is then inserted into the proximal end of outer cannula 4. Applying a suitable force on inner cannula handle 26, the distal end 22 of the cannula is forced into the intravertebral or cancellous material. The inner beveling of the distal end of the inner cannula facilitates penetration of the distal end into the intravertebral material. As distal portion 32 further penetrates into the vertebral material, a core of material is urged into the lumen of the inner cannula. The initial diameter of the core has a diameter the size of the diameter at distal edge 36. As the inner diameter tapers radially inward, the cored material is compressed and compacted so as to fit into the smaller diameter at proximal edge 34 and into the lumen of inner cannula 8. As such, the density of the cored material is increased thereby providing a tighter fit of the cored material within the lumen and thereby more securely retaining the cored material within the lumen upon removal of inner cannula 8. The cored material is then removed from inner cannula 8 and analyzed as a biopsy sample or is otherwise disposed of if solely removed for the purpose of creating a space within the target site. The biopsy or coring procedure may be repeated as many time as necessary, i.e., until a sufficient amount of sample is obtained or until the desired size of the cored area is achieved. The same inner cannula or other inner cannulas may be used to obtain additional cores.

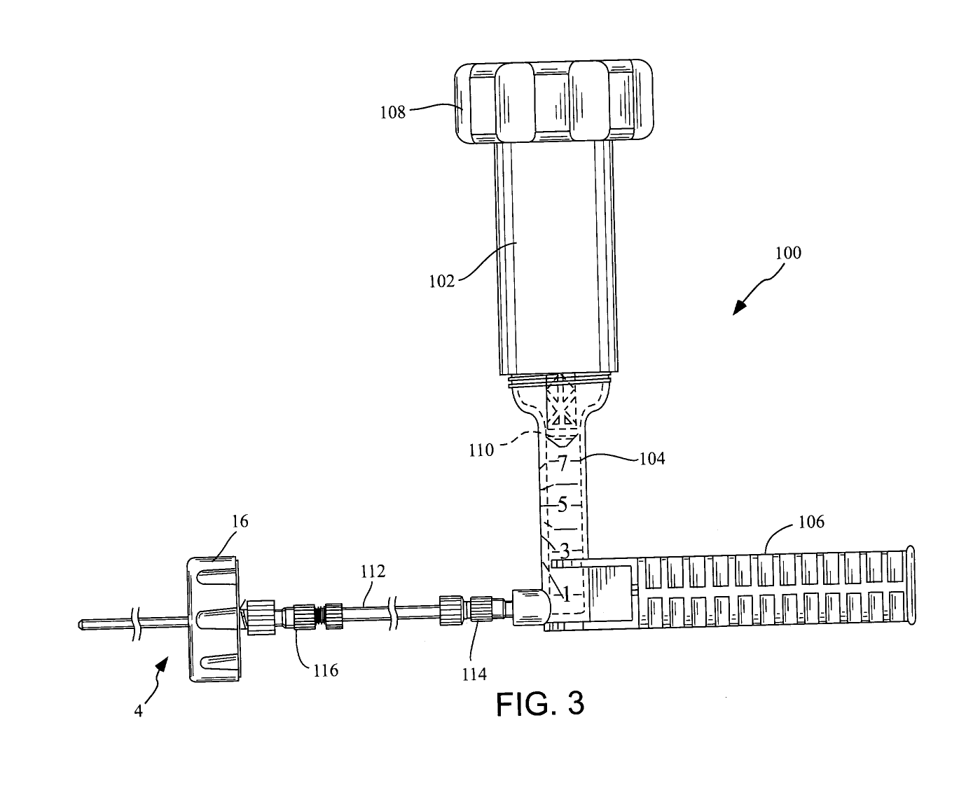

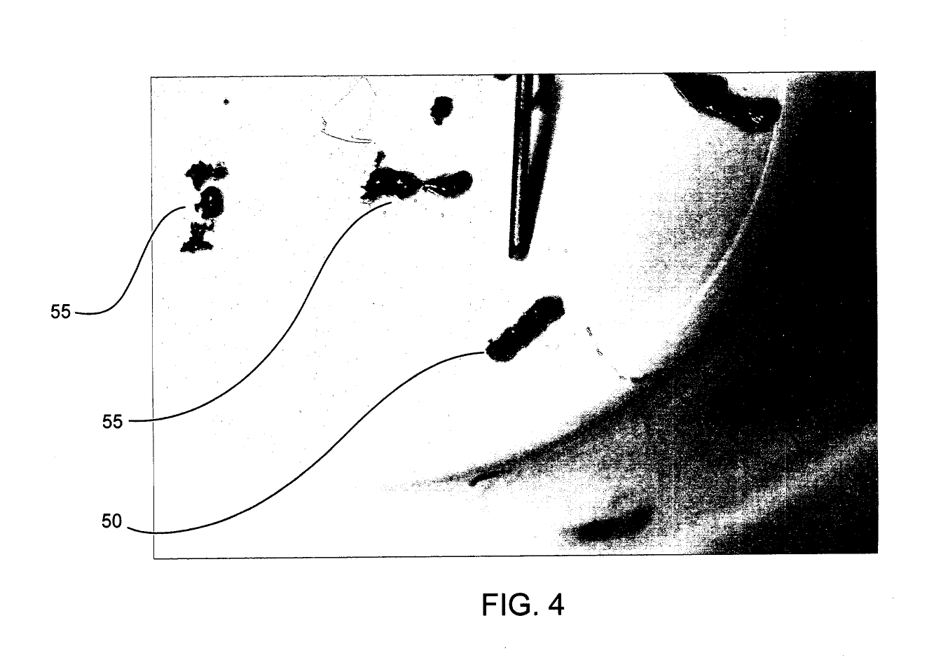

For applications involving the delivery of a cement or filler material to within the target access site, inner cannula 8 is removed from outer cannula 4 which is left in place at the target site. A system 100 for the controlled injection of filler material is operatively coupled to outer cannula 4, as shown in FIG. 3, so as to be in fluid communication with the cannula's lumen. System 100 generally includes a first column 102 and a second column 104 which holds the filler material. A handle 108 at the proximal end of first column 102 is used to drive and pressurize the filler material through column 102 and into the second column 104. Extending distally from first column 102 is a plunger head 110 for forcing the filler material through the second column 104. System 100 is in fluid communication with outer cannula 4 by means of a tubing 112 which is interconnected to system 100 and cannula 4 by luer locks 114 and 116, respectively. A handle 106 is provided for manually handling system 100. Once system 100 is properly connected to outer cannula 4, the filler material is delivered to within the space created by the coring process described above until a selected amount of such filler material has been injected into the space. Upon completion of the filling process, the system 100 is disconnected from outer cannula 4 which is then removed from the access site, and the wound site is treated with typical care. U.S. patent application Ser. No. 09/408,690, herein incorporated by reference, further describes such a system 100 and the manner in which it is employed with the present invention.

Kits

Also provided by the present invention are kits that include the devices as described above. The kits may include a plurality of outer cannulas, inner cannulas and stylets for use in a variety of applications.

In addition, the subject kits typically include instructions for using the subject systems in methods according to the subject invention. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the Internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

An experiment was conducted in order to validate the ability of the inner cannula's inner beveled tip design to compress and retain tissue samples, and to show improvement over prior art devices which an outer beveled distal tip. The prior art device used is the Parallax ClearView™ 11 Ga by 11.6 cm needle. The experiment involved the removal of cancellous tissue from various vertebrae of a cadaver by both the present invention and the prior art device. FIG. 3 illustrates various cored samples in which the sample referenced as 50 was extracted by the subject inner cannula and those referenced as 55 are samples taken by the prior art cannula, respectively. As is clear from the photograph, the cored sample 50 is intact and has more cohesion than cored samples 55. More specifically, the cored sample 50 has remained in a solid formation having relatively defined length and diameter dimensions as compared to those samples 55 cored with the prior art device. As such, the cored sample 50 is more suitable for analysis.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the present invention and the appended claims. That being said,

What is claimed is:

1. A medical device, comprising:
a cannula having a length defining a longitudinal axis, an outer diameter, an inner diameter, a proximal end and an inwardly beveled distal tip extending from the outer diameter to the inner diameter,
wherein the outer diameter is constant along the length from the proximal end to the distal tip and the inner diameter is constant along the length from the proximal end up to but not including the distal tip, and
wherein the distal tip defines a plane at least substantially normal to the longitudinal axis.

2. The medical device of claim 1 wherein the inwardly beveled distal tip is funnel-shaped.

3. The medical device of claim 1 wherein the inwardly beveled distal tip is frustroconical-shaped.

4. A medical device, comprising:
a cannula having a proximal end and a distal portion having a proximal edge and a distal edge,
wherein the cannula has a constant outer diameter from the proximal end to the distal edge, and
wherein the wall thickness is constant from the proximal end to the proximal edge of the distal portion and tapers inwardly from the distal edge to the proximal edge.

5. The medical device of claim 4 wherein the distal portion defines a space in the shape of a right frustrum.

6. A medical device, comprising:
a cannula comprising a smooth, inviolate inner luminal surface, an outer luminal surface, and an inwardly beveled distal portion having a substantially circular distal edge, wherein the inner luminal and outer luminal surfaces are parallel.

7. The medical device of claim 6 wherein the beveled distal portion defines an angle in the range from about 5° to about 60°.

8. The medical device of claim 7 wherein the beveled distal portion defines an angle of about 15°.

9. The medical device of claims 1, 4 or 6, further comprising a second cannula configured for slidably receiving the cannula.

10. The medical device of claim 9, further comprising a stylet configured for slidable engagement within the second cannula.

11. A system for carrying out medical procedures at a target site within the body, comprising:
a first cannula;
a second cannula configured for slidable engagement within said second cannula and having a length defining a longitudinal axis, an outer diameter, an inner diameter, a proximal end and an inwardly beveled distal tip, wherein the outer diameter is constant along the length from the proximal end to the distal tip and the inner diameter is constant along the length from the proximal end up to but not including the distal tip, and wherein the distal tip defines a plane normal to the longitudinal axis; and
a stylet configured for slidable engagement within the first cannula.

12. The system of claim 11 wherein the first cannula is configured for delivering a flowable material to within the body.

13. A system for carrying out medical procedures at a target site within the body, comprising:
an outer cannula having a handle at a proximal end and configured at a distal end for penetration into the target site;
an inner cannula configured for slidable engagement within the outer cannula and having a handle at a proximal end configured for locking engagement with the handle of the outer cannula and having an inwardly tapered distal tip defining a plane normal to the longitudinal axis of the inner cannula; and
a stylet configured for slidable engagement within the outer cannula and having a handle at a proximal end configured for locking engagement with the handle of the outer cannula.

14. The system of claim 13 wherein the outer cannula handle is configured for fluid engagement with a device for the controlled injection of a filler material.

15. A medical procedure to be performed on a body, comprising:
providing the system of claim 13;
slidably engaging the stylet within the outer cannula;
penetrating tissue with said assembled stylet and outer cannula and advancing the assembling to a target site;
removing the stylet form the outer cannula and slidably engaging the inner cannula within the outer cannula; and
extracting tissue from the target site with the inner cannula thereby defining a space with the target site.

16. The medical procedure of claim 15, further comprising:
repeating the extracting of tissue as necessary;

17. The medical procedure of claim 15, further comprising:
filling the space within the target site with a flowable material.
